(19) United States
(12) Patent Application Publication
(10) Pub. No.: US 2022/0298470 A1
(43) Pub. Date: Sep. 22, 2022

(54) MYCELIUM-BASED MATERIALS INCLUDING HIGH-PERFORMANCE INSULATION AND RELATED METHODS

(71) Applicant: BIOHM Ltd, London (GB)

(72) Inventors: Ehab Sayed, London (GB); Antonius Marie Driessen, London (GB); Samantha Gini Rebecca Jenkins, London (GB); Candyce-Robyne Dryburgh, London (GB); Francesco Verderosa, London (GB); Irene Li Barnett, London (GB)

(73) Assignee: BIOHM Ltd, London (GB)

(21) Appl. No.: 17/678,285

(22) Filed: Feb. 23, 2022

(30) Foreign Application Priority Data
Feb. 24, 2021 (GB) 2102595.2

(51) Int. Cl. C12N 1/14; C12N 13/00

(52) U.S. Cl. CPC C12N 1/14; C12N 13/00

(57) ABSTRACT

Mycelium-based materials including high-performance insulation and related methods are generally described.

### TECHNICAL FIELD

Mycelium-based materials including high-performance insulation and related methods are generally described.

### SUMMARY

Mycelium-based materials including high-performance insulation and related methods are described herein. The articles may include mycelium-based materials that include one or more components of mycelium. The subject matter of the present disclosure involves, in some cases, interrelated products, alternative solutions to a particular problem, and/or a plurality of different uses of one or more systems and/or articles.

In one aspect, a method is described, the method comprising culturing mycelium to produce a first grain spawn, culturing the first grain spawn to produce a mother spawn, and colonizing a target substrate with the mother spawn. In some embodiments, colonizing comprises producing a first mixture of carbon dioxide and water, flowing the first mixture to a container comprising a photosynthetic species, producing oxygen with the photosynthetic species, flowing the oxygen to the mother spawn, and producing a second mixture of carbon dioxide and water, and flowing the second mixture to the container comprising the photosynthetic species. In some embodiments, the method further comprises deactivating any mycelium.

In another aspect, a method is described, the method comprising culturing mycelium to produce a first grain spawn, culturing the first grain spawn to produce a mother spawn, colonizing a target substrate with the mother spawn, and deactivating the mother spawn and/or any mycelium. In some embodiments, any one of the culturing steps comprises feeding mycelium a nutritional material that has been treated with a mixture of an acid and a peroxide.

In another aspect, a method is described, the method comprising culturing mycelium to produce a first grain spawn, culturing the first grain spawn to produce a mother spawn, colonizing a target substrate with the mother spawn, wherein colonizing comprises applying an electric field to at least a portion of the mother spawn and/or the target substrate, and deactivating the mother spawn and/or any mycelium.

In a different aspect, a mycelium-based material is described, the mycelium-based material comprising a plurality of hyphal filaments, wherein a wt % of chitin within the hyphal filaments is greater than or equal to 5 wt %, and a target substrate mixed with the plurality of hyphal filaments, wherein the thermal conductivity of the mycelium-based material is less than or equal to 0.03 W/m·K, wherein the heat of combustion of the mycelium-based material is less than or equal to 30 MJ/kg, and wherein a porosity of the mycelium-based material is greater than or equal to 1%.

In another aspect, a mycelium-based material is described, the mycelium-based material comprising a plurality of hyphal filaments, wherein a wt % of chitin within the hyphal filaments is greater than or equal to 5 wt %, a target substrate mixed with the plurality of hyphal filaments, wherein the thermal conductivity of the mycelium-based material is less than or equal to 0.1 W/m·K, wherein the heat of combustion of the mycelium-based material is less than or equal to 17 MJ/kg, and wherein a porosity of the mycelium-based material is greater than or equal to 1%.

In yet another aspect, a mycelium-based material is described, comprising a plurality of hyphal filaments, and a target substrate mixed with the plurality of hyphal filaments, wherein the wt % of target substrate in the mycelium-based material is greater than or equal to 1 wt %, wherein the thermal conductivity of the mycelium-based material is less than or equal to 0.1 W/m·K, wherein the heat of combustion of the mycelium-based material is less than or equal to 30 MJ/kg, and wherein a porosity of the mycelium-based material is greater than or equal to 1%.

In yet another aspect, a mycelium-based material is described, comprising a plurality of hyphal filaments, wherein the plurality of hyphal filaments is arranged to form a disordered network, and a target substrate mixed with the plurality of hyphal filaments, wherein the thermal conductivity of the mycelium-based material is less than or equal to 0.1 W/m·K, wherein the heat of combustion of the mycelium-based material is less than or equal to 30 MJ/kg, and wherein a porosity of the mycelium-based material is greater than or equal to 1%.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B are schematic illustrations of the growth of one or more grain spawns starting from a culture of mycelium, according to some embodiments; and

FIG. 2 shows a schematic illustration of a system for applying current to mycelium, according to some embodiments.

### DETAILED DESCRIPTION

Methods and articles comprising mycelium are described herein. Mycelium forms the vegetative root portion of a fungus and may have branching, thread-like hyphae that can extend into a substrate. The methods described herein may be used to prepare materials that comprise mycelium. The mycelium may be used to provide beneficial properties (e.g., structural support, low thermal conductivity) to mycelium-based materials. Accordingly, mycelium-based materials may include mycelium or one or more components from mycelium (e.g., hyphal filaments, mycelium cell walls). The mycelium is used to grow a material from a target substrate, and the living mycelium within the material may, in some embodiments, be subsequently deactivated so that the mycelium-based material contains no living mycelium while comprising components from the mycelium.

The methods and articles described herein may have several advantages over certain existing methods for producing mycelium-based materials. As one advantage, the mycelium described herein may be configured (e.g., cultured) to decompose (or at least partially decompose) a target substrate, such as polymers or plastics, which have been difficult to decompose using certain existing methods. In some cases, the target substrate may be at least partially or completely consumed by the mycelium.

As another advantage, the mycelium (i.e., the hyphae of the mycelium) may be tailored, adapted to, or otherwise configured to decompose a particular target substrate. For example, the mycelium may produce harder or stiffer hyphae to penetrate the target substrate if the target substrate is relatively hard and/or non-porous. The mycelium may also produce compounds (e.g., enzymes) that further facilitate decomposing of a particular target substrate. In some cases, the resulting mycelium-based material may benefit from decomposing a particular target substrate. For example, when mycelium decomposes a relatively hard target substrate, the resulting hyphae may be structurally more robust or contain higher concentrations of chitin within the hyphal filaments of the mycelium when compared to decomposing a softer target substrate. This advantage can be used to tune or tailor the resulting mycelium-based material to have a particular property (e.g., hardness, thermal conductivity, fire resistance, heat resistance).

The mycelium may grow by feeding on a substrate (e.g., a target substrate). A substrate in this context is any material that mycelium is capable of digesting or decomposing, or any other nutritious feedstock that can be inoculated with mycelium to produce mycelium-based materials. The substrate can be a variety of suitable materials. In some embodiments, the substrate comprises lignocellulosic material. In some embodiments, the substrate comprises a biomaterial, such as hemp, flax, cotton, cork, sheep's wool, wood, and/or coconut fibers, without limitation. In some embodiments, the substrate comprises a non-lignocellulosic material, such as synthetic materials. In some embodiments, the substrate comprises a plastic or a polymer. Non-limiting examples of suitable polymers include polyethylene (e.g., low-density polyethylene, high-density polyethylene), polypropylene, polyurethane, and/or polystyrene. Those skilled in the art in view of the teachings of the present disclosure will be capable of selecting suitable substrates for the mycelium.

In some embodiments, the substrate (e.g., target substrate) has a particular hardness. The hardness of the substrate may be measured using Young's elastic modulus. In some embodiments, the substrate has a Young's elastic modulus of greater than or equal to 1 GPa, greater than or equal to 5 GPa, greater than or equal to 10 GPa, greater than or equal to 20 GPa, greater than or equal to 30 GPa, greater than or equal to 40 GPa, greater than or equal to 50 GPa, greater than or equal to 75 GPa, greater than or equal to 100 GPa, greater than or equal to 110 GPa, greater than or equal to 120 GPa, or greater than or equal to 130 GPa. In some embodiments, the substrate has a Young's elastic modulus of less than or equal to 130 GPa, less than or equal to 120 GPa, less than or equal to 110 GPa, less than or equal to 100 GPa, less than or equal to 75 GPa, less than or equal to 50 GPa, less than or equal to 40 GPa, less than or equal to 30 GPa, less than or equal to 20 GPa, less than or equal to 10 GPa, less than or equal to 5 GPa, or less than or equal to 1 GPa. Combinations of the above-referenced ranges are also possible (e.g., greater than or equal to 1 GPa and less than or equal to 130 GPa).

As mentioned above, a target substrate can be selected to impart one or more desired physical and/or chemical properties to the mycelium-based material. In some embodiments, the mycelium completely consumes the target substrate in order to produce the mycelium-based material. In other embodiments, however, the target substrate is only partially consumed, such that the resulting mycelium-based material contains at least some of the target substrate. In consuming the target substrate, the mycelium-based material may take on desired properties as a result of the target substrate, such as an increased concentration (e.g., mass percent, weight percent, mole fraction) of a structural component within the mycelium (e.g., hyphal filaments) such as chitin, polysaccharides, or another chemical species of the mycelium. In some embodiments, consuming the target substrate may change (e.g., increase) an amount of a non-structural component, such as the amount of protein or other mycelium metabolite within the mycelium-based material. Those skilled in the art in view of the teachings of the present disclosure will be able to determine if the target substrate is to be completely consumed or if the target substrate is only partially consumed such that the mycelium-based end product comprises at least some target substrate.

To produce mycelium-based materials, a culture of mycelium is developed, which can be used to inoculate or colonize a target substrate. Accordingly, methods described herein may comprise culturing mycelium. In some embodiments, the mycelium cultures are maintained in their exponential growth phase so as to achieve a relatively high growth rate of the mycelium. A variety of culturing techniques are known. For example, the mycelium may be cultured using Petri dishes or liquid medium (i.e., a liquid culture) containing nutritional materials (e.g., malt extract, yeast). The mycelium may be grown to select for certain properties (e.g., growth rate, metabolism) that may be useful in fabricating mycelium-based materials. In some embodiments, a single species of mycelium is cultured. In other embodiments, however, more than one species of mycelium can be cultured.

The mycelium can be cultured under conditions suitable to facilitate the growth of the mycelium. For example, the growth temperature of the mycelium may be controlled and at a temperature of greater than or equal to 4° C., greater than or equal to 5° C., greater than or equal to 6° C., greater than or equal to 7° C., greater than or equal to 8° C., greater than or equal to 9° C., greater than or equal to 10° C., greater than or equal to 15° C., greater than or equal to 20° C., greater than or equal to 21° C., greater than or equal to 22° C., greater than or equal to 23° C., greater than or equal to 24° C., greater than or equal to 25° C., greater than or equal to 26° C., greater than or equal to 27° C., greater than or equal to 28° C., greater than or equal to 29° C., or greater than or equal to 30° C. In some embodiments, the growth temperature of the culturing mycelium is less than or equal to 30° C., less than or equal to 29° C., less than or equal to 28° C., less than or equal to 27° C., less than or equal to 26° C., less than or equal to 25° C., less than or equal to 24° C., less than or equal to 23° C., less than or equal to 22° C., less than or equal to 21° C., less than or equal to 20° C., less than or equal to 15° C., less than or equal to 10° C., less than or equal to 9° C., less than or equal to 8° C., less than or equal to 7° C., less than or equal to 6° C., less than or equal to 5° C., or less than or equal to 4° C. Combinations of the above-referenced ranges are also possible (e.g., greater than or equal to 20° C. and less than or equal to 30° C.). Other ranges are possible.

In some embodiments, the mycelium may produce heat as it cultures and grows and, in some such embodiments, this heat may be used to passively maintain temperatures in incubation spaces without the need for external heaters, which may reduce cost and energy usage in culturing the mycelium. However, in some embodiments, a heater (and/or air conditioner, such as those used in HVAC systems) may be used to adjust the growth temperature of the mycelium. In some embodiments, one or more sensors may be used to determine the growth temperature of the mycelium (i.e., to determine the temperature of the environment the mycelium are cultured in) and may be configured (e.g., via connection to a heater and/or air conditioner) to adjust the temperature to a desired growth temperature.

In some cases, classical strain breeding may be applied to the culturing mycelium so that mycelium with properties suited for a particular application amongst different wild type strains (e.g., non-bred strains) may grow in order to develop unique strains that are tailored for the desired application of mycelium-based material. In some such embodiments, variables such as lignocellulose degradation, spawn run speed (i.e., colonization speed), and/or resilience to other microbes may be beneficial genetic traits to use for selection.

In some embodiments, basidiospores (a reproductive spore produced by at least some mycelium) are collected, from which monokaryons (offspring) are grown. Compatible mating types may then be combined to form dikaryotic mycelium, which may have twice the amount of genetic information available to them. These offspring may be tested on a target substrate. In some embodiments, the isolates that favorably interact with the target substrate may be selected, combined, and may be continually subjected to a repetitive breeding cycle, eventually resulting in accumulation of beneficial mutations and speciation of the mycelium. Specimen differentiation and evolution can be confirmed in antagonism experiments and random amplified polymorphic DNA analysis.

A culture of mycelium may be further developed by allowing the culture of mycelium to inoculate a nutritional “grain” for the mycelium to digest. In digesting the grain, the population of the mycelium increases and allows the “grain spawn” to expand in size. These grain spawns can be grown successively, wherein a portion of a first grain is taken to populate a second grain of the mycelium to inoculate. However, it should be understood the mycelium may inoculate nutritional material other than grain (e.g., a complex nutritional substrate), as this disclosure is not so limited.

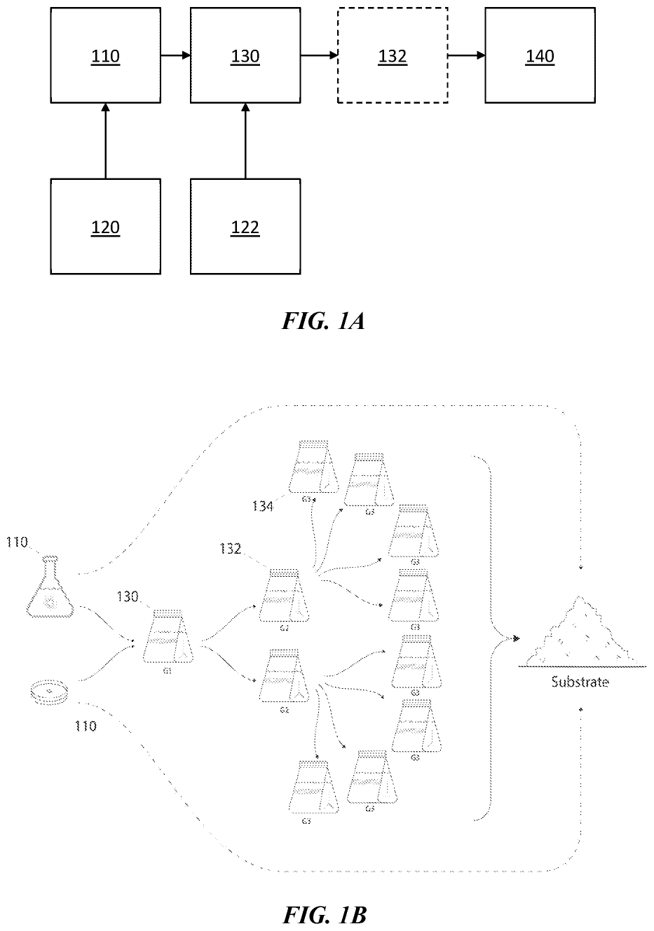

FIG. 1A shows a flow diagram demonstrating the production of grains from an initial culture of mycelium. In the figure, mycelium 110, from a culture of mycelium, is provided with nutritional material 120 (e.g., “grain”) in order to produce a first grain spawn 130, which comprises mycelium grown from the culture of mycelium 110. The nutritional material 120 may comprise compounds that facilitate the growth of the mycelium (e.g., grain, feedstock, malt extract, yeast), in addition other components (e.g., enzymes, enzyme production enhancers).

Mycelium grown within the first grain spawn may be used to produce additional mycelium in a second grain spawn. For example, as schematically illustrated in FIG. 1A, the first grain spawn 130 is provided with additional nutritional material 122 to produce a second grain spawn 132, which comprises newly grown mycelium from the first grain spawn 130. While the same nutritional material used to produce the first grain spawn may have the same material composition as the nutritional material used to produce the second grain spawn, in some cases the nutritional material is different. That is to say, in some embodiments, nutritional material 120 and 122 may be the same; however, in other embodiments, nutritional material 120 and 122 are different. As shown illustratively in this figure, in some embodiments the second grain spawn (or the first grain spawn) may be used to produce a mother spawn 140, as described in more detail below. The mother spawn may comprise mycelium that can be used to colonize a substrate (e.g., a target substrate).

The nutritional material may be or comprise nutritional substances that the mycelium can use to feed upon and grow. In some cases, the nutritional material may become more complex from the first grain spawn to subsequent grain spawns (e.g., the second grain spawn, a third grain spawn, a fourth grain spawn). For example, the nutritional material used to feed an initial culture of mycelium may comprise relatively simple nutritional material (e.g., malt extract, yeast, simple carbohydrates) while subsequent nutritional materials may comprise relatively more complex (e.g., harder to digest) nutritional material (e.g., plant-based substrates, grasses, grains, seeds, husks, wood-based waste streams). Without wishing to be bound by any particular theory, the presence of harder to digest nutrients may have an effect on the cellular composition of the mycelium, for example, shifting to higher concentrations of lipids, proteins, and chitin. Furthermore, with more complex and/or harder to digest nutritional material (or substrate), the mycelium's hyphal tips may become stiffer and tougher as an adaptation to the pressure from penetrating the harder or more complex nutritional material. In some cases, these compositional changes may advantageously affect the quality of the mycelium-based material (e.g., rigidity, hydrophobicity, incombustibility). In some embodiments, the nutritional material comprises at least some target substrate such that the mycelium may be exposed to the target substrate during its growth to develop or aid in developing the desired properties of the mycelium-based material

As used herein, grain refers to a feedstock used to grow the mycelium or feedstock that is inoculated by the mycelium. Accordingly, a “grain spawn” is a mixture of nutritional material (e.g., grain) and mycelium. The first grain spawn is produced from an initial amount of mycelium (e.g., from a mycelium culture) and nutritional material. In some embodiments, additional grain spawns can be produced using the first grain spawn or the second grain spawn. For example, the first grain spawn (or at least a portion of the first grain spawn, e.g., 10 wt %) can be used to produce the second grain spawn, the second grain spawn can be used to produce a third grain spawn, the third grain spawn can be used to produce a fourth grain spawn, and so forth. Each subsequent grain spawn may include at least a portion of mycelium from the previous grain spawn. For example, in FIG. 1B, mycelium culture 110 mixed with a feedstock to produce a first grain spawn 130. Subsequently, at least a portion of the first grain spawn 130 is used to produce a second grain spawn 132, and at least a portion of the second grain spawn 132 is used to produce a third grain spawn 134.

In some embodiments, a grain spawn is formed by using greater than or equal to 1 wt %, greater than or equal to 2 wt %, greater than or equal to 3 wt %, greater than or equal to 4 wt %, greater than or equal to 5 wt %, greater than or equal to 7 wt %, greater than or equal to 10 wt %, greater than or equal to 12 wt %, greater than or equal to 15 wt %, greater than or equal to 18 wt %, greater than or equal to 20 wt %, greater than or equal to 25 wt %, or greater than or equal to 30 wt % of the desired total amount the grain spawn. In some embodiments, a grain spawn is formed by using less than or equal to 30 wt %, less than or equal to 25 wt %, less than or equal to 20 wt %, less than or equal to 18 wt %, less than or equal to 15 wt %, less than or equal to 12 wt %, less than or equal to 10 wt %, less than or equal to 7 wt %, less than or equal to 5 wt %, less than or equal to 4 wt %, less than or equal to 3 wt %, less than or equal to 2 wt %, or less than or equal to 1 wt % of the desired total amount of the grain spawn. Combinations of the above-referenced ranges are also possible (e.g., greater than or equal to 1 wt % and less than or equal to 30 wt %). Other ranges are possible. As a hypothetical, illustrative example, if 100 g of a second grain spawn is desired, then 10 g (i.e., 10 wt % of 100 g) of the first spawn may be used to form the second spawn, while the remaining 90 g comprises nutritional material or other components (e.g., enzyme production enhancers, mediators)

In some embodiments, the nutritional material undergoes a pasteurization process (e.g., prior to the nutritional material being introduced to the mycelium). This process may be used to remove or reduce the amount of any potential pathogens or contaminants that could inhibit the growth of the mycelium. A variety of suitable pasteurization processes may be used. For example, in some embodiments, the nutritional material is steam pasteurized. In some embodiments, the nutritional material is treated with an autoclave. In some embodiments, the nutritional material is chemically pasteurized, for example, using hydrogen peroxide as described in more detail below.

In some embodiments, the nutritional material may comprise or be treated with a mixture of acids and/or peroxides (e.g., prior to the nutritional material being introduced to the mycelium). That is to say, culturing may comprise feeding mycelium a nutritional material that has been treated with a mixture of an acid and a peroxide. Acids or peroxides, independently or in combination, may be used to reduce or minimize microorganism within the nutritional material, similar to other pasteurization processes (e.g., steam pasteurization). The use of acids and/or peroxides to treat the nutritional material may be particularly advantageous when relatively large quantities of nutritional material are to be treated, as the solution of acids and/or peroxide may allow for better degradation of the material and promote nutritional availability of nutrients to the mycelium.

The combination of acid and peroxide may be particularly advantageous. In some embodiments, the combination of acid with peroxide (e.g., hydrogen peroxide) may increase the oxidizing ability of the hydrogen peroxide. Without wishing to be bound by any particular theory, their combination may produce reactive oxygen species, in addition to heat, which may enhance the reduction of the amount of microorganisms present in the nutritional material relative to using the individual acid or peroxide separately. In some cases, once the nutritional material has been treated with the mixture of acid and/or peroxide, the mixture may be neutralized or rendered chemically inert such that the mixture does not inhibit or inactivate the mycelium present in the grain spawn or mother spawn.

In some embodiments, the mixture of acid and peroxide comprises citric acid and/or hydrogen peroxide. However, a variety of acids and/or peroxides may be used in the mixture. Non-limiting examples of acids include organic acids (e.g., citric acid, acetic acid), phosphoric acid, hydrochloric acid, nitric acid, and/or sulfuric acid. Non-limiting examples of peroxides include hydrogen peroxide (e.g., H₂O₂ in 30 wt % H₂O), organic peroxides (e.g., benzoyl peroxide), or other peroxides.

In some embodiments, the concentration of peroxide (e.g., hydrogen peroxide) within a solution used to treat the nutritional material (or substrate) is greater than or equal to 1 wt %, greater than or equal to 3 wt %, greater than or equal to 5 wt %, greater than or equal to 7 wt %, greater than or equal to 10 wt %, greater than or equal to 15 wt %, greater than or equal to 20 wt %, greater than or equal to 30 wt %, or greater than or equal to 35 wt % of the solution. In some embodiments, the concentration of peroxides of a solution used to treat the nutritional material (or substrate) is less than or equal to 10 wt %, less than or equal to 7 wt %, less than or equal to 5 wt %, less than or equal to 3 wt %, or less than or equal to 1 wt % of the solution. Combinations of the above-referenced ranges are also possible (e.g., greater than or equal to 1 wt % and less than or equal to 10 wt %). Other ranges are possible.

In some embodiments, the pKₐ of an acid used is less than or equal to 5, less than or equal to 4, less than or equal to 3, less than or equal to 2, less than or equal to 1, less than or equal to 0, less than or equal to −1, less than or equal to −2, or less than or equal to −3. In some embodiments, the pKₐ of the acid is greater than or equal to −3, greater than or equal to −2, greater than or equal to −1, greater than or equal to 0, greater than or equal to 1, greater than or equal to 2, greater than or equal to 3, greater than or equal to 4, or greater than or equal to 5. Combinations of the above-referenced ranges are also possible (e.g., greater than or equal to 0 and less than or equal to 4). Other ranges are possible.

Culturing may also comprise providing enzyme production enhancers to a grain spawn (e.g., a first grain spawn, a second grain spawn). Enzyme production enhancers may increase the amount of enzymes produced by the mycelium which can improve their growth or assist the mycelium in breakdown of a substrate. In some embodiments, enzyme production enhancers can be included to upregulate expression of the genes that are suited for digestion of a target substrate. Without wishing to be bound by any particular theory, the enzyme production enhancers may increase transcription of the enzymes of interest (e.g., lignin-digesting enzymes like laccase and peroxidase) and can be added during a grain spawn phase (e.g., a first grain spawn, a second grain spawn, a third grain spawn) before the target substrate is introduced. In some embodiments, the enzyme production enhancer comprises metal ions or salts thereof (e.g., a copper salt), ammonium salts, sulfate salts, peptone water, and/or organic compounds (e.g., xylidines).

In some embodiments, the mycelium are cultured in the presence of mediators. As used in this context, a mediator is an organic compound that can be oxidized by enzymes produced by the mycelium (e.g., laccase) to form, without wishing to be bound by any particular theory, high reactive cation radicals capable of oxidizing non-phenolic compounds that laccase alone cannot oxidize. In some embodiments, the mediator comprises NHPI (N-hydro-xyphthalimide), VLA (violuric acid (5-isonitrosobarbituric acid)), TEMPO (2,2′,6,6′-tetramethylpiperidine) TMAH (tetramethylammoniumhydroxide), and/or NHA (N-hydroxy-N-phenylacetamide).

In some embodiments, the first grain spawn or the second grain spawn may be used to produce a mother spawn. The mother spawn may comprise mycelium that is tailored to colonize one or more target substrates after being developed from a grain spawn. For example, as shown schematically in FIG. 1A, the second grain spawn 132 produces mother spawn 140. However, it should be understood that in some embodiments, the mother spawn 140 may be produced from the first grain spawn 130. That is to say, in some cases, the second grain spawn 132 and/or any subsequent grain spawns may be optional. And while not shown in the figure, some embodiments may have one or more additional grain spawns (e.g., a third grain spawn, a fourth grain spawn, a fifth grain spawn), which may give rise to the mother spawn. Those skilled in the art in view of the teachings of the present disclosure will be capable of determining the number of grain spawns used to produce the mother spawn.

The mother spawn comprises mycelium that can be used to colonize a substrate (e.g., a target substrate). In some embodiments, the substrate may be colonized with a plate culture of mycelium or a liquid culture of mycelium. In some embodiments, colonizing comprises mixing the mycelium with the target substrate. In colonizing a target substrate, the mycelium of the mother spawn may (at least partially) break down or decompose the target substrate as the mycelium grows, producing new mycelium with properties related to or derived from the target substrate. In some embodiments, the mycelium only partially decomposes the target substrate, so that at least some of the initial target substrate is present in the resultant mycelium-based material or product. In some embodiments, the amount of target substrate within the mycelium-based material is greater than or equal to 1 wt %, greater than or equal to 3 wt %, greater than or equal to 5 wt %, greater than or equal to 10 wt %, greater than or equal to 15 wt %, greater than or equal to 20 wt %, greater than or equal to 25 wt %, greater than or equal to 30 wt %, greater than or equal to 40 wt %, greater than or equal to 50 wt %, greater than or equal to 60 wt %, or greater than or equal to 70 wt % relative to the total weight of the mycelium-based material. In some embodiments, the amount of target substrate within the mycelium-based material is less than or equal to 70 wt %, less than or equal to 60 wt %, less than or equal to 50 wt %, less than or equal to 40 wt %, less than or equal to 30 wt %, less than or equal to 25 wt %, less than or equal to 20 wt %, less than or equal to 15 wt %, less than or equal to 10 wt %, less than or equal to 5 wt %, less than or equal to 3 wt %, or less than or equal to 1 wt % relative to the total weight of the mycelium-based material. Combinations of the above-referenced ranges are also possible (e.g., greater than or equal to 1 wt % and less than or equal to 70 wt %). Other ranges are possible. However, in other embodiments, the target substrate is significantly consumed by the mycelium such that little (e.g., less than 1 wt %, less than or equal to 0.1 wt %) to no target substrate is present in the resultant mycelium-based material or product.

In some cases, the mycelium consumes oxygen (e.g., oxygen gas) as they colonize or grow, producing water and carbon dioxide as byproducts. In some such embodiments, the carbon dioxide and water produced by the mycelium may be flowed to one or more photosynthetic organisms, which may, in turn, consume the carbon dioxide and water to produce oxygen that may be flowed back to the mycelium. In this way, a circular loop or closed system may be maintained in which the mycelium provide carbon dioxide and water to a photosynthetic species, and the photosynthetic species provides oxygen to the mycelium or a derivative thereof, which can produce more carbon dioxide to provide to the photosynthetic species. In such embodiments, this system may advantageously reduce or eliminate emissions of carbon dioxide from the mycelium and may also help control or regulate the environment of the mycelium. Accordingly, in some embodiments, colonizing comprises, producing a first mixture of carbon dioxide and water, flowing the first mixture to a container and/or controlled environment comprising a photosynthetic species (e.g., a greenhouse), producing oxygen with the photosynthetic species, flowing the oxygen to the mycelium (which, in some embodiments, may be contained in an incubation room), producing a second mixture of carbon dioxide and water, and/or flowing the second mixture of carbon dioxide and water to the container and/or controlled environment comprising the photosynthetic species. In some embodiments, the closed loop may be maintained in a continuous or circular manner, such that the mycelium and the photosynthetic species continuously provide one another with carbon dioxide/water and oxygen.

In some embodiments, an electrical current or an electrical field may be applied prior to, during, or after culturing the mycelium. Electricity may generally enhance the growth of mycelium during culturing the mycelium or colonizing a substrate. In some embodiments, colonizing comprises applying an electric field to at least a portion of the mother spawn and/or the target substrate. Without wishing to be bound by any particular theory, mycelium may spontaneously grow in a radial manner from point or central location. However, it has been recognized within the context of the present disclosure, that applying a current or electric field to the mycelium may interrupt or disrupt the radial growth of the mycelium so that the arrangement or orientation of the mycelium within the mycelium-based material may be modified (e.g., tuned, controlled, shaped) during growth. This may be advantageously used to control the growth direction of the mycelium which may be used to selectively control the orientation of the mycelium in the mycelium-based material. In some embodiments, the application of current may also increase the rate of growth of the mycelium. Additionally, in some embodiments, applying an electric field or current may be used to control the growth direction of the mycelium such that it requires no additional molding or shaping (e.g., growing the mycelium-based material within a mold).

An electric field or an electric current may be applied in any suitable manner. For example, in some embodiments, electrodes connected to a potentiostat may be used to apply a voltage to the mycelium. In some embodiments, a potential of greater than or equal to 1 V, greater than or equal to 10 V, greater than or equal to 20 V, greater than or equal to 25 V, greater than or equal to 50 V, greater than or equal to 100 V, greater than or equal to 250 V, greater than or equal to 500 V, greater than or equal to 1 kV, greater than or equal to 2 kV, greater than or equal to 3 kV, greater than or equal to 4 kV, greater than or equal to 5 kV, or greater than or equal to 10 kV is applied to the mycelium (e.g., during culturing, during colonizing). In some embodiments, a potential of less than or equal to 10 kV, less than or equal to 5 kV, less than or equal to 4 kV, less than or equal to 3 kV, less than or equal to 2 kV, less than or equal to 1 kV, less than or equal to 500 V, less than or equal to 250 V, less than or equal to 100 V, less than or equal to 50 V, less than or equal to 25, V, less than or equal to 20 V, less than or equal to 10 V, or less than or equal to 1 V. Combinations of the above-referenced ranges are also possible (e.g., greater than or equal to 1 V and less than or equal to 10 V applied to the mycelium). Other ranges are possible.

In some embodiments, the voltage or electric current applied to the mycelium is pulsed (e.g., a 1 second pulse, a 10 second pulse, a 1-minute pulse, a 10-minute pulse). In some such embodiments, the orientation of the mycelium is disordered or randomized by pulsing the electric current, which may advantageously strengthen the resulting mycelium-based material.

In some embodiments, the mycelium may be deactivated or otherwise rendered biologically inactive. In this way, the mycelium-based material comprises relatively little or no living mycelium. While relatively little to no living mycelium may be present in the mycelium-based material, components of the mycelium (e.g., hyphal filaments, proteins, lipids) may remain in the mycelium-based material. It should also be understood that the growth orientation of the mycelium (e.g., as controlled by an electric current or field) may remain intact even after deactivating the mycelium. In some embodiments, the mycelium is deactivated during or after colonizing a substrate (e.g., a target substrate). In some embodiments, deactivating the mycelium may also aid in curing the mycelium-based material.

In some embodiments, deactivating the mycelium comprises a first deactivation step, and, optionally, a second deactivation step and/or additional deactivation steps. The first activation step may at least partially deactivate the mycelium (e.g., at a relatively lower temperature), while a subsequent deactivation step may more completely deactivate the mycelium (e.g., at a relatively higher temperature). A variety of techniques may be used to deactivate the mycelium, some of which are described below in more detail.

In some embodiments, deactivating the mycelium comprises heating and/or dehydrating the mycelium. Any suitable technique may be used to heat and/or dehydrate the mycelium, such as a vacuum pump, a drying oven, a vacuum oven, a heater, chemical dehydrating agents, as non-limiting examples.

In some embodiments, deactivating the mycelium (e.g., a first deactivation step) comprises heating the mycelium to a temperature of greater than or equal to 40° C., greater than or equal to 50° C., greater than or equal to 60° C., greater than or equal to 70° C., greater than or equal to 80° C., greater than or equal to 90° C., or greater than or equal to 100. In some embodiments, deactivating the mycelium comprises heating the mycelium to a temperature of less than or equal to 100° C., less than or equal to 90° C., less than or equal to 80° C., less than or equal to 70° C., less than or equal to 70° C., less than or equal to 50° C., or less than or equal to 40° C. Combinations of the above-referenced ranges are also possible (e.g., greater than or equal to 40° C. and less than or equal to 100° C.). Other ranges are possible.

In some embodiments, deactivating the mycelium (e.g., a first deactivation step, a second deactivation step) comprises heating the mycelium to a temperature of greater than or equal to 110° C., greater than or equal to 120° C., greater than or equal to 130° C., greater than or equal to 140° C., greater than or equal to 150° C., greater than or equal to 160° C., greater than or equal to 170° C., greater than or equal to 180° C., greater than or equal to 190° C., greater than or equal to 200° C., greater than or equal to 210° C., or greater than or equal to 220° C. In some embodiments, deactivating the mycelium comprises heating the mycelium to a temperature of less than or equal to 220° C., less than or equal to 210° C., less than or equal to 200° C., less than or equal to 190° C., less than or equal to 180° C., less than or equal to 170° C., less than or equal to 160° C., less than or equal to 150° C., less than or equal to 140° C., less than or equal to 130° C., less than or equal to 120° C., or less than or equal to 110° C. Combinations of the above-referenced ranges are also possible (e.g., greater than or equal to 110° C. and less than or equal to 180° C.). Other ranges are possible.

In some embodiments, deactivating the mycelium comprises applying a radiofrequency (e.g., microwaves) to the mycelium. In some cases, applying a radiofrequency to the mycelium thermally activates the mycelium (or one or more components within or adjacent to the mycelium, such as the nutritional material) while avoiding exposing the mycelium to direct heat. Any suitable method to provide the radiofrequency may be used.

In some embodiments, deactivating the mycelium comprises lyophilization (i.e., freeze drying). Lyophilization can be accomplished, for example, using a laboratory or commercial lyophilizer.

As mentioned above, mycelium may be used to produce mycelium-based materials that comprise one or more components of the mycelium (e.g., hyphal filaments). The mycelium-based material may be a final product, for example, to be sold or used as insulation in houses or buildings or may be a precursor to a final product that may be further processed (e.g., dried).

In some embodiments, the mycelium-based material comprises a plurality of hyphal filaments. The hyphal filaments provide support and structure to the mycelium and may be used to provide support and structure, along with other properties, to mycelium-based materials even after the mycelium has been deactivated. In some embodiments, the hyphal filaments may be mixed with the target substrate, which may also help provide support to the mycelium-based materials. In some cases, the hyphal filaments contain compounds such as chitin and/or polysaccharides, which may impart desired properties to the mycelium-based material. In some embodiments, the plurality of hyphal filaments remains intact or undisrupted within the mycelium-based material. In other embodiments, however, at least a portion of the contents of the mycelium hyphal filaments may be disrupted such that the compounds within the hyphae are dispersed within the mycelium-based material.

In some embodiments, the mycelium-based material has a particular weight percentage of chitin within the hyphal filaments or within the mycelium-based material. In some embodiments, a wt % of chitin is greater than or equal to 1 wt %, greater than or equal to 3 wt %, greater than or equal to 5 wt %, greater than or equal to 10 wt %, greater than or equal to 12 wt %, greater than or equal to 15 wt %, greater than or equal to 18 wt %, greater than or equal to 20 wt %, greater than or equal to 25 wt %, greater than or equal to 30 wt %, greater than or equal to 40 wt %, or greater than or equal to 50 wt % relative to the total weight of the mycelium-based material. In some embodiments, a wt % of chitin is less than or equal to 50 wt %, less than or equal to 40 wt %, less than or equal to 30 wt %, less than or equal to 25 wt %, less than or equal to 20 wt %, less than or equal to 18 wt %, less than or equal to 15 wt %, less than or equal to 12 wt %, less than or equal to 10 wt %, less than or equal to 5 wt %, or less than or equal to 3 wt %, or less than or equal to 1 wt % relative to the total weight of the mycelium-based material. Combinations of the above-referenced ranges are also possible (e.g., greater than or equal to 1 wt % and less than or equal to 50 wt %). Other ranges are possible. In some embodiments, chitin within the mycelium-based material (e.g., within the plurality of hyphal filaments) may provide the material with improved fire-resistant, such as a relatively low thermal conductivity and/or improved heats of combustion. The wt % of chitin in the material can be determined using a chitin assay. A chitin assay can be performed by a deproteinization and/or a deacetylation of the mycelium-based material, followed by depolymerizing the mycelium-based material to produce glucosamine and using a glucosamine assay to indirectly determine the amount of chitin in the mycelium-based material. Those of ordinary skill in the art will be capable of selecting an appropriate glucosamine assay for determining the amount of chitin in a mycelium-based material.

In some embodiments, a particular amount of water is present within the mycelium-based material. In some embodiments, the amount of water present within the mycelium-based materials is less than or equal to 20 wt %, less than or equal to 15 wt %, less than or equal to 12 wt %, less than or equal to 10 wt %, less than or equal to 7 wt %, less than or equal to 5 wt %, less than or equal to 3 wt %, or less than or equal to 1 wt % relative to the total weight of the mycelium-based material. In some embodiments, the amount of water present within the mycelium-based material is greater than or equal to 1 wt %, greater than or equal to 3 wt %, greater than or equal to 5 wt %, greater than or equal to 7 wt %, greater than or equal to 10 wt %, greater than or equal to 12 wt %, greater than or equal to 15 wt %, or greater than or equal to 20 wt % relative to the total weight of the mycelium-based material. Combinations of the above-referenced ranges are also possible (e.g., greater than or equal to 1 wt % and less than or equal to 20 wt %). Other ranges are possible.

In some embodiments, the mycelium-based material may have a particular thermal conductivity. In some embodiments, the thermal conductivity of the mycelium-based material is less than or equal to 0.1 W/m·K, less than or equal to 0.09 W/m·K, less than or equal to 0.08 W/m·K, less than or equal to 0.07 W/m·K, less than or equal to 0.06 W/m·K, less than or equal to 0.05, W/m·K, less than or equal to 0.04 W/m·K, less than or equal to 0.03 W/m·K, or less than or equal to 0.02 W/m·K. In some embodiments, the thermal conductivity of the mycelium-based material is greater than or equal to 0.02 W/m·K, greater than or equal to 0.03 W/m·K, greater than or equal to 0.04 W/m·K, greater than or equal to 0.05 W/m·K, greater than or equal to 0.06 W/m·K, greater than or equal to 0.07 W/m·K, greater than or equal to 0.08 W/m·K, greater than or equal to 0.09 W/m·K, or greater than or equal to 0.1 W/m·K. Combinations of the above-referenced ranges are also possible (e.g., greater than or equal to 0.02 W/m·K and less than or equal to 0.03 W/m·K). Other ranges are possible. The thermal conductivity can be measured via a steady state technique using ASTM Standard Test C 177-63.

The mycelium-based materials described herein may have a particular heat of combustion. In some embodiments, the heat of combustion of the mycelium-based material is less than or equal to 30 MJ/kg, less than or equal to 25 MJ/kg, less than or equal to 20 MJ/kg, less than or equal to 15 MJ/kg, less than or equal to 10 MJ/kg, less than or equal to 8 MJ/kg, less than or equal to 5 MJ/kg, less than or equal to 3 MJ/kg, less than or equal to 2 MJ/kg, or less than or equal to 1 MJ/kg. In some embodiments, the heat of combustion of the mycelium-based material is greater than or equal to 1 MJ/kg, greater than or equal to 2 MJ/kg, 3 MJ/kg, greater than or equal to 5 MJ/kg, greater than or equal to 8 MJ/kg, greater than or equal to 10 MJ/kg, greater than or equal to 15 MJ/kg, greater than or equal to 20 MJ/kg, greater than or equal to 25 MJ/kg, or greater than or equal to 30 MJ/kg. Combinations of the above-referenced ranges are also possible (e.g., less than or equal to 30 MJ/kg and greater than or equal to 1 MJ/kg). Other ranges are possible. In some embodiments, the heat of combustion of the mycelium-based material may be achieved absent or without the application of a chemical coating or treatment. The heat of combustion of the material can be determined using a standard test, such as EN ISO standard test 1716:2018.

In some embodiments, the mycelium-based material has a particular porosity (e.g., an average porosity). In some embodiments, the mycelium-based material has a porosity of less than or equal to 70%, less than or equal to 60%, less than or equal to 50%, less than or equal to 40%, less than or equal to 35%, less than or equal to 30%, less than or equal to 25%, less than or equal to 20%, less than or equal to 15%, less than or equal to 10%, less than or equal to 5%, or less than or equal to 1%. In some embodiments, the mycelium-based material has a porosity of greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 50%, greater than or equal to 60%, or greater than or equal to 70%. Combinations of the above-referenced ranges are also possible (e.g., greater than or equal to 1% and less than or equal to 70%). Other ranges are possible. The porosity and pore size distribution can be measured via mercury intrusion porosimetry using a standard test, such as standard test BS ISO 15901-01:2016.

The pores of mycelium-based material may be of any suitable size (e.g., an average cross-sectional pore diameter). In some embodiments, the pores have an average cross-sectional diameter of greater than or equal to 1 nm, greater than or equal to 2 nm, greater than or equal to 5 nm, greater than or equal to 10 nm, greater than or equal to 20 nm, greater than or equal to 30 nm, greater than or equal to 40 nm, greater than or equal to 50 nm, greater than or equal to 75 nm, greater than or equal to 100 nm, greater than or equal to 250 nm, greater than or equal to 500 nm, greater than or equal to 1 micron, greater than or equal to 10 microns, greater than or equal to 20 microns, greater than or equal to 25 microns, greater than or equal to 50 microns, greater than or equal to 100 microns, greater than or equal to 200 microns, greater than or equal to 300 microns, greater than or equal to 400 microns, or greater than or equal to 500 microns. In some embodiments, the pores have an average cross-sectional diameter of less than or equal to 500 microns, less than or equal to 400 microns, less than or equal to 300 microns, less than or equal to 200 microns, less than or equal to 100 microns, less than or equal to 50 microns, less than or equal to 25 microns, less than or equal to 20 microns, less than or equal to 10 microns, less than or equal to 1 micron, less than or equal to 500 nm, less than or equal to 250 nm, less than or equal to 100 nm, less than or equal to 75 nm, less than or equal to 50 nm, less than or equal to 40 nm, less than or equal to 30 nm, less than or equal to 20 nm, less than or equal to 10 nm, less than or equal to 5 nm, or less. Combinations of the above-referenced ranges are also possible (e.g., greater than or equal to 1 nm and less than or equal to 100 nm). Other ranges are possible.

In some embodiments, the hyphal filaments of the mycelium-based material may be fused and/or crosslinked. Without wishing to be bound by any theory, as the mycelium colonizes and grows within a target substrate, its hyphal tips may extend and populate the target substrate as the mycelium-based material is formed. In some cases, the hyphal tips may become crosslinked amongst each other, which may advantageously improve the properties of the mycelium-based material (e.g., improved mechanical strength, improved fire resistance). In some cases, the hyphal tips may make contact with other hyphal tips of the mycelium and may fuse to one another. Advantageously, fusion of hyphal filaments may also improve the properties of the mycelium-based material.

In some embodiments, the hyphal filaments (e.g., the plurality of hyphal filaments) may have a particular orientation within the mycelium-based material. For example, in some embodiments, the hyphal filaments may have a parallel or antiparallel arrangement. However, in some embodiments, the hyphal filaments have no particular orientation and may be ordered in a random or disordered manner, such as tortuous network.

The mycelium-based material may be any suitable thickness. In some embodiments, the mycelium-based material has a thickness of greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm, greater than or equal to 15 mm, greater than or equal to 20 mm, greater than or equal to 25 mm, greater than or equal to 30 mm, greater than or equal to 40 mm, greater than or equal to 50 mm, greater than or equal to 60 mm, greater than or equal to 70 mm, greater than or equal to 75 mm, greater than or equal to 80 mm, greater than or equal to 90 mm, greater than or equal to 100 mm, greater than or equal to 110 mm, greater than or equal to 120 mm, greater than or equal to 130 mm, greater than or equal to 140 mm, or greater than or equal to 150 mm. In some embodiments, mycelium-based material has a thickness of less than or equal to 150 mm, less than or equal to 140 mm, less than or equal to 130 mm, less than or equal to 120 mm, less than or equal to 110 mm, less than or equal to 1 cm, less than or equal to 90 mm, less than or equal to 80 mm, less than or equal to 75 mm, less than or equal to 70 mm, less than or equal to 60 mm, less than or equal to 50 mm, less than or equal to 40 mm, less than or equal to 30 mm, less than or equal to 25 mm, less than or equal to 20 mm, less than or equal to 15 mm, less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, or less than or equal to 1 mm. Combinations of the above-referenced ranges are also possible (e.g., greater than or equal to 1 mm and less than or equal to 30 mm). Other ranges are possible.

In some embodiments, the mycelium-based material is free of any coatings. Without wishing to be bound by any particular theory, at least some of the beneficial, advantageous properties of the mycelium-based materials of the present disclosure may be achieved without applying a coating to the material. In some such embodiments, this may improve the properties of the material, such as improved air flow through the material (relative to a mycelium-based material with an applied coating). However, it should be understood that, in some embodiments, a coating is present, as this disclosure is not so limited.

In some embodiments, the mycelium-based materials described herein may be formed into any suitable shape. In some such embodiments, the mycelium-based material may be a freeform material, wherein the material does not conform to a regular shape or structure. In some embodiments, an electric field or current may be used to shape the mycelium (e.g., into a regular shape, into a freeform shape). For example, in some embodiments, the mycelium-based material may be formed or shaped by the application of a current to the growing mycelium-material, which may be used to direct the growth or orientation of the mycelium of the material. In some embodiments, a container or tray may be used to form or help form the mycelium-based material into a particular shape. For example, the mycelium (e.g., a mother spawn) may be cultured or colonized in a container or tray, so that the mycelium-based material has the shape of the container or tray. Those skilled in the art in view of the teachings of the present disclosure will be capable of shaping the mycelium-based material for a desired application.

The methods and articles described herein may be used for any number of suitable applications. One exemplary application includes using the mycelium-based materials as insulating materials in construction settings, such as for homes and buildings. In some embodiments, the mycelium-based materials described herein may have relatively low thermal conductivities, which may make these materials particularly useful as insulating materials. Because the materials are created by living mycelium, the resulting bio-based insulation materials may be seen as an environmentally conscious alternative to synthetic materials (e.g., plastics).

In some embodiments, the mycelium-based materials described herein may be useful as fire-retardant or flame-resistant materials. In some cases, the mycelium-based materials have a relatively low heat of combustion, which may improve the fire-retardant properties of the material.

The following examples are intended to illustrate certain embodiments of the present invention, but do not exemplify the full scope of the invention.

### EXAMPLE

The following example describes the preparation of mycelium-based materials starting with culturing the mycelium through to the deactivation of the living mycelium in the mycelium-based materials.

### Culturing

This stage was the starting point of the mycelium culture. A fresh plate transfer was made, grown on a rich, agar medium and later transferred either into grain or onto a new plate. A variety of nutritional materials can be used. Plates were used for transferring or storing cultures.

### Plate Cultures

Plate transfers were performed aseptically by transferring a tissue sample from the parent plate to a new plate using either a hollow needle or small cubes of agar cut using a scalpel. The plates were incubated at a temperature, 21-30° C., and a humidity-controlled environment was used when an appropriate growth rate of the mycelium was reached.

### Liquid Cultures

Liquid cultures were also used to culture the mycelium. Mycelium could be grown in liquid medium from either a plate transfer, cryostock, or from another liquid culture and later transferred either into more complex substrate or into new medium. A variety of nutritional materials can be used. The liquid culture could provide more even dispersal of the mycelium throughout the substrate and could further be homogenized by blending or mixing for an even greater dispersal when poured onto the substrate.

To prepare a liquid culture, a bottle of liquid medium was sterilized. Once sterilized, the bottle was inoculated with the mycelium of the chosen strain in order to colonize the substrate. When used for inoculating substrate, an amount of the medium and the mycelium was measured out and poured onto the substrate before mixing with the substrate.

### Grain Spawn

Mycelium from a plate or liquid culture was transferred to grain (in a grain container). In some cases, a previously established grain was further expanded to a larger grain container. A variety of grains can be used or a mix, such as those described elsewhere herein, without limitation. The grain was hydrated, sterilized, and inoculated with colonized grain spawn or culture from a plate or liquid. The samples were incubated in a controlled environment at temperatures between 21-30° C.

### Plate or Liquid to Grain Transfer

In a sterile environment, the mycelium from plates or liquid cultures was transferred to the prepared grain, mixed thoroughly, stored in an incubation room at an appropriate temperature at temperatures between 21-30° C. and the progress observed.

### Grain to Grain Expansion Using a Portion of Colonized Grain to Inoculate Fresh, Sterilized Grain

In a sterile environment an amount of colonized grain spawn is transferred into the new grain (e.g., 2-40 wt % of the fresh grain) was weighed and transferred into a new container of grain and repeated until all containers were colonized.

### Pre-Processing of Substrate: Treatment/Sterilization+Inoculation Process

In this process, the substrate (e.g., agricultural waste streams) was treated so as to sterilize the substrate and subsequently inoculated in preparation for the mycelium to grow.

In some cases, a steam pasteurization method was implemented. The substrate was treated with steam and the heating cycle was considered complete after the core temperature of the substrate had reached or surpassed 65-95° C. for 5-30 minutes depending on specific application and substrate in question. The substrate was then cooled before inoculation by the mycelium of the grain spawn.

In some cases, peroxides were also used to sterilize the substrate in lieu or in combination with steam pasteurization. The desired amount of dry substrate to be treated was chemically sterilized with a diluted peroxide solution.

### Growth of Mycelium-Based Materials

Pasteurized and Inoculated Substrates were Shaped into Desired Shapes, or Sculpted into Desired Forms

The pasteurized and inoculated was placed in an incubation room and grown until the substrate was colonized. An HVAC system could be used to control the ambient humidity in the incubation room, with a humidity of 60-95%. The temperature of each incubation space was kept at an appropriate level for the strain being used.

### Electric Current Applied to Mycelium

In some cases, electricity was used to increase the growth of the mycelium and/or to alter the growth direction or orientation of the mycelium. Once the pasteurized and inoculated substrate was ready for incubation, a voltage of up to 6 kV could be applied to the mycelium for set periods of time at different intervals and the growth monitored. The time scale is inversely proportional to voltage. For example, a period of minutes was used when applying voltages near 6 kV at an interval of every three to five days.

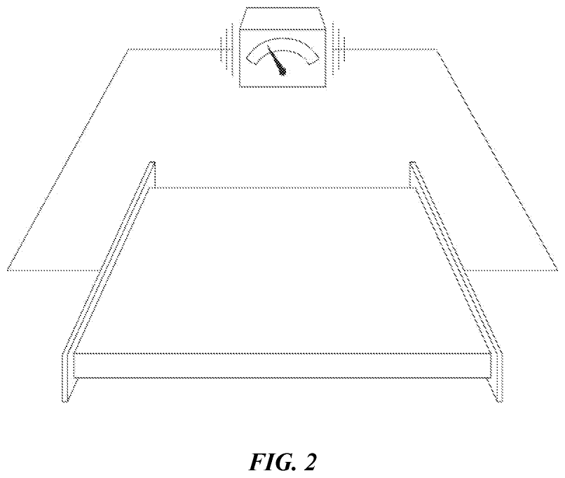

A schematic illustration is shown in FIG. 2. The setup can readily be reconfigured to accommodate a variety of sizes and shapes.

### Deactivating the Mycelium

Living mycelium within the mycelium-based material was deactivated. One or two processes to deactivate the mycelium once the samples finished growing were used: (1) Dehydration using a low temperature over a long period of time and (2) Dehydration using higher temperatures for a shorter amount of time. The size of the mycelium-based material determined the amount of time needed for it to be fully dried. A temperature reading of an interior portion of the mycelium-based material could be obtained using a temperature probe and heating was discontinued once the temperature reached a desired value.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
culturing mycelium to produce a first grain spawn;
culturing the first grain spawn to produce a mother spawn;
colonizing a target substrate with the mother spawn, wherein colonizing comprises:
producing a first mixture of carbon dioxide and water,
flowing the first mixture to a container comprising a photosynthetic species, producing oxygen with the photosynthetic species,
flowing the oxygen to the mother spawn, and producing a second mixture of carbon dioxide and water and flowing the second mixture to the container comprising the photosynthetic species; and
deactivating any mycelium.

2. A method, comprising:
culturing mycelium to produce a first grain spawn;
culturing the first grain spawn to produce a mother spawn;
colonizing a target substrate with the mother spawn; and
deactivating the mother spawn and/or any mycelium,
wherein any one of the culturing steps comprises feeding mycelium a nutritional material that has been treated with a mixture of an acid and a peroxide.

3. A method, comprising:
culturing mycelium to produce a first grain spawn;
culturing the first grain spawn to produce a mother spawn;
colonizing a target substrate with the mother spawn, wherein colonizing comprises applying an electric field to at least a portion of the mother spawn and/or the target substrate; and
deactivating the mother spawn and/or any mycelium.

4-7. (canceled)

8. The method of claim 1, wherein a wt % of chitin within hyphal filaments of the mycelium is greater than or equal to 5 wt % and/or less than or equal to 95 wt %.

9. The method of claim 1, wherein the target substrate is greater than or equal to 5 wt % of the mycelium-based material.

10-12. (canceled)

13. The method of claim 1, wherein an amount of water present in the mycelium-based material is less than or equal to 20 wt % after the deactivating step.

14. (canceled)

15. The method of claim 1, wherein a Young's modulus of the target substrate is greater than or equal to 1 GPa and/or less than or equal to 130 GPa.

16. (canceled)

17. The method of claim 1, wherein culturing comprises growing a first grain spawn, a second grain spawn, and/or a third grain spawn.

18. The method of claim 1, wherein culturing comprises producing a mother spawn.

19. The method of claim 2, wherein the mixture of acid and peroxide comprises citric acid and/or hydrogen peroxide.

20. The method of claim 1, wherein culturing comprises providing enzyme production enhancers.

21. The method of claim 1, wherein colonizing comprises mixing the mycelium with the target substrate.

22. The method of claim 1, further comprising forming the mycelium and/or the substrate into shape.

23. The method of claim 1, wherein:
(a) deactivating comprises dehydrating and/or heating the mycelium, and/or
(b) deactivating comprises applying a radiofrequency to the mycelium, and/or
(c) deactivating comprises lyophilizing the mycelium, and/or
(d) deactivating comprises a first deactivation step, and, optionally, a second deactivating step.

24. The method of claim 1, wherein colonizing the target substrate comprises a liquid culture of mother spawn.
